(19) United States
(12) Patent Application Publication
(10) Pub. No.: US 2002/0048594 A1
(43) Pub. Date: Apr. 25, 2002

(54) Rosmarinus extracts for inhibiting/treating skin aging

(76) Inventors: Lionel Breton, Versailles (FR); Francoise Bernerd, Paris (FR); Richard Martin, Rochecorbon (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/904,560

(22) Filed: Jul. 16, 2001

Related U.S. Application Data
(63) Continuation of application No. PCT/FR00/00034, filed on Jan. 10, 2000.

(30) Foreign Application Priority Data
Jan. 15, 1999 (FR) 99/00406

(51) Int. Cl. A61K 7/06; A61K 35/78

(52) U.S. Cl. 424/401; 424/745

(57) ABSTRACT

The adverse/deleterious signs or effects of skin aging, for example, wrinkles and fine lines, withered skin, flabby skin, thinning of the dermis, etc., are inhibited/treated by topically applying onto the skin, hair and/or mucous membranes of an individual subject in need of such treatment, a thus effective amount of at least one extract of at least one plant of the Rosmarinus genus, or cosmetic/dermatological composition comprised thereof.

### CROSS-REFERENCE TO PRIORITY/PCT APPLICATIONS

This application claims priority under 35 U.S.C. §119 of FR-99/00406, filed Jan. 15, 1999, and is a continuation of PCT/FR00/00034, filed on Jan. 10, 2000 and designating the United States (published in the French language on Jul. 20, 2000 as WO 00/41674; the title and abstract were also published in English), both hereby expressly incorporated by reference.

### BACKGROUND OF THE INVENTION

1. Technical Field of the Invention

The present invention relates to topical application onto human skin and/or mucous membranes of at least one extract of at least one plant of the Rosmarinus genus for treating, preventively and/or curatively, cutaneous signs of aging.

The present invention also relates to novel compositions comprising such extracts to stimulate the restructuring of, and/or inhibit damage to, the skin and/or mucous membranes by stimulating the synthesis of collagen and/or inhibiting collagenases. This invention, thus, also relates to a regime/regimen for the cosmetic treatment of the skin and/or mucous membranes.

2. Description of the Prior Art

Human skin is composed of two compartments or layers, namely, a surface compartment or layer, the epidermis, and a deep compartment or layer, the dermis.

The natural human epidermis is composed principally of three types of cells, which are keratinocytes, melanocytes and Langerhans cells, the vast majority being keratinocytes. Each of these cell types contributes, via its specific functions, to the essential role played by the skin in the body.

The dermis provides the epidermis with a solid support. It is also its source of nutrition. It is composed principally of fibroblasts and of an extracellular matrix, itself composed principally of collagen, of elastin and of a substance referred to as ground substance, which components are synthesized by the fibroblast. It also comprises leukocytes, mastocytes or tissue macrophages. It is also traversed by blood vessels and nerve fibers. In normal skin, i.e., nonpathological and noncicatricial, the fibroblast is in the quiescent state, i.e., nonproliferative, not very active from a metabolic point of view and nonmobile.

It is the collagen fibers which provide the dermis with strength. The collagen fibers are composed of fibrils, which are firmly attached to one another, thus defining more than ten types of different structure. The strength of the dermis is in large part due to the entanglement of the collagen fibers, which are packed tightly against one another in all directions. The collagen fibers contribute to the elasticity and to the tonicity of the skin and/or mucous membranes.

The collagen fibers are constantly replaced, but this replacement decreases with age, which results in thinning of the dermis. This thinning of the dermis is also due to pathological causes, such as, for example, the hypersecretion of corticoid hormones, certain diseases (Marfan syndrome, Ehlers-Danlos syndrome) or vitamin deficiencies (scurvy). It is also accepted that extrinsic factors, such as ultraviolet rays, tobacco or certain treatments (glucocorticoids, vitamin D and derivatives, for example), also have an effect on the skin and on its level of collagen.

However, various factors cause damage to the collagen, with all the conceivable consequences regarding the structure and/or the firmness of the skin and/or mucous membranes.

Although highly resistant, collagen fibers are sensitive to certain enzymes known as collagenases. Damage to the collagen fibers results in the appearance of flabby and wrinkled skin which humans, preferring the appearance of a smooth and taut skin, have always sought to combat.

Collagenases belong to a family of enzymes known as metalloproteinases (MMPs) which are, themselves, members of a family of proteolytic enzymes (endoproteases) which have a zinc atom coordinated to three cysteine residues and a methionine in their active site, and which degrade the macromolecular components of the extracellular matrix and the basal laminae at neutral pH (collagen, elastin, etc). Very widely distributed in the body, these enzymes are present, but weakly expressed, in normal physiological conditions such as organ growth and tissue renewal.

Their overexpression in humans and their activation are, however, related to many processes, sometimes pathological processes, which entail the destruction and remodelling of the matrix. This results in either uncontrolled resorption of the extracellular matrix or, on the other hand, the setting up of a state of fibrosis.

The metalloproteinase family is composed of several well defined groups based on their similarities in terms of structure and of substrate specificity (see Woessner J. F., Faseb Journal, 5, 2145 (1991)). Among these groups, exemplary are collagenases intended to degrade fibrillar collagens (MMP-1 or interstitial collagenase, MMP-8 or neutrophil collagenase, or MMP-13 or collagenase 3), gelatinases which degrade type IV collagen or any form of denatured collagen (MMP-2 or gelatinase A (72 kDa), or MMP-9 or gelatinase B (92 kDa)), stromelysins (MMP-3), the broad spectrum of activity of which applies to proteins of the extracellular matrix, such as glycoproteins (fibronectin, laminin), proteoglycans, etc., or membrane-bound metalloproteinases.

Prolonged exposure to ultraviolet radiation, particularly to type A and/or B ultraviolet radiation, has the effect of stimulating the expression of collagenases, particularly of MMP-1. This is one of the manifestations of photoinduced cutaneous aging.

Furthermore, at menopause, the principal modifications relating to the dermis are a decrease in the level of collagen and in dermal thickness. In menopausal women, this promotes thinning of the skin and/or mucous membranes. Women then experience a “dry skin” or tight skin feeling, and an accentuation of surface fine wrinkles and fine lines is noted. The skin has a rough appearance on palpation. Finally, the skin exhibits reduced suppleness.

From the foregoing, the importance of collagen in the structure of tissues, particularly of the skin and/or mucous membranes, will be appreciated, as will the importance of not only maintaining its level in the tissues, but also of combating damage thereto, such as to thus control aging, whether chronobiological or photoinduced aging, and its consequences, the thinning of the dermis and/or the damage to collagen fibers, which results in the appearance of flabby and wrinkled skin.

### SUMMARY OF THE INVENTION

Accordingly, a major object of the present invention is the provision of unique active agents/species having a stimulatory effect on collagen synthesis, an inhibitory effect on collagenases and, to the extent possible, no significant side effects.

Briefly, it has now surprisingly and unexpectedly been determined that an extract of at least one plant of the Rosmarinus genus has stimulatory activity with regard to collagen synthesis and inhibitory activity with regard to collagenase activity.

### DETAILED DESCRIPTION OF BEST MODE AND SPECIFIC/PREFERRED EMBODIMENTS OF THE INVENTION

More particularly according to the present invention, plants of the Rosmarinus genus, other than their gustative and aromatic properties, are known for their anti-inflammatory effects. Compare, for example, FR-A-2,504,551, JPA7017846, WO-A-9325209, SU-A-1733000, FR-A-2,662,078, DE-A-3536342, U.S. Pat. No. 5,393,526 or WO-A-9701288 (assigned to the assignee hereof).

Nonetheless, it is believed that, to date, the stimulatory activity with regard to collagen synthesis, and the inhibitory activity with regard to collagenase activity, of an extract of at least one plant of the Rosmarinus genus have never been described.

Thus, the present invention, in a first embodiment thereof, features formulating at least one extract of at least one plant of the Rosmarinus genus into compositions suited for treating, preventively and/or curatively, cutaneous signs of aging.

By the expression “extract of at least one plant of the Rosmarinus genus” are intended both a crude mixture of parts of the plant roughly cut up into pieces and of the extraction solvent, and developed preparations of the active principles solubilized during the extraction.

By the term “active principle” is intended any molecule or composition capable of modifying or of modulating the functioning of at least one given biological system.

By the expression “cutaneous signs of aging” is intended any modifications of the external appearance of the skin due to aging, whether chronobiological and/or photoinduced, such as, for example wrinkles and fine lines, withered skin, flabby skin, thinned skin or lack of elasticity and/or of tone of the skin, but also any internal modifications of the skin which are not systematically reflected by a modified external appearance, such as, for example, any internal damage to the skin, particularly to the collagen, resulting from exposure to ultraviolet radiation.

In another embodiment of the invention, at least one extract of at least one plant of the Rosmarinus genus is administered to stimulate collagen synthesis.

In yet another embodiment of the invention, at least one extract of at least one plant of the Rosmarinus genus is administered to inhibit the expression of extracellular matrix proteases, particularly metalloproteinases and even more particularly type 1 metalloproteinase.

This invention also features administration of at least one extract of at least one plant of the Rosmarinus genus to treat cutaneous conditions/afflictions related to menopause.

This invention also features administration of at least one extract of at least one plant of the Rosmarinus genus to combat skin wrinkles and fine lines.

In another embodiment of the invention, at least one extract of at least one plant of the Rosmarinus genus is administered to an individual in need of such treatment, to combat withered skin.

In another embodiment of the invention, at least one extract of at least one plant of the Rosmarinus genus is administered to combat flabby skin.

This invention also features administration of at least one extract of at least one plant of the Rosmarinus genus to combat thinned skin.

And this invention also features topical application of at least one extract of at least one plant of the Rosmarinus genus, or composition comprised thereof, to combat lack of elasticity and/or of tone of the skin.

The extract of at least one plant of the Rosmarinus genus according to the invention can be obtained from plant material derived from a whole plant, or from a part of a plant, such as the leaves, the stems, the flowers, the petals or the roots, or even from dedifferentiated cells.

By the expression “dedifferentiated plant cells” is intended any plant cell which has none of the characteristics of a particular specialization and which is capable of surviving alone and in a situation where it is not dependent on other cells.

Preferably, according to the invention, the whole plant is used, particularly stem and/or leaves, very particularly leaves.

The extract of at least one plant of the Rosmarinus genus can be any extract prepared from any plant material derived from at least one plant of the Rosmarinus genus cultivated in vivo or derived from culturing in vitro.

By the expression “cultivating in vivo” is intended any cultivation of conventional type, i.e., in soil, in the open air or under glass, or, alternatively, soilless.

And by the expression “culturing in vitro” are intended all of the techniques known to this art which make it possible to artificially obtain a plant or a part of a plant. The selection pressure imposed by the physicochemical conditions during the growth of the plant cells in vitro makes it possible to obtain a standardized plant material which is available throughout the year, unlike plants cultivated in vivo.

Preferably according to the invention, a plant of the Rosmarinus genus derived via cultivating in vivo is employed.

The Rosmarinus officinalis species comprises two varieties, which are Rosmarinus tenuifolius and Rosmarinus latifolius.

The extracts of the invention can be prepared from plant material originating from the Rosmarinus officinalis species, whatever the variety.

Any extraction technique known to the art can be employed to prepare the extract according to the invention.

Exemplary, in particular, are alcoholic extractions, especially ethanolic or aqueous/alcoholic extractions.

Preferably, the extract is an aqueous extract.

It is also envisaged to use an extract prepared by the method described in French patent application No. 95-02379, assigned to the assignee hereof. Thus, in a first step, the plant material is ground in an aqueous solution while cold and, in a second step, the particles in suspension are removed from the aqueous solution derived from the first step. This aqueous solution corresponds to the extract. Optionally, in a third step, the aqueous solution derived from the second step is sterilized. This extract can then be lyophilyzed.

The first step may be advantageously replaced with a simple procedure for freezing the plant tissues (for example at 20° C., or alternatively at 180° C. in liquid nitrogen), followed by an aqueous extraction repeating the second and third steps described above.

The cold treatment makes it possible to freeze the enzymatic activities, and the sterilizing filtration avoids degradation of the active agents by the microorganisms of the environment. Finally, the water vehicle is compatible with ex vivo receptors and facilitates cosmetic or pharmaceutical formulations.

It is known that plant extracts contain oxidases responsible, inter alia, for the oxidation of said extracts. Such an oxidation produces a dark brown coloration of the extracts and a pungent smell, rendering same relatively incompatible with use in cosmetics. Along these lines, a laccase, the molecular weight of which is greater than 100,000 daltons, is, in particular, known.

Thus, advantageously, the extract obtained can be fractionated by any known fractionation method which makes it possible to eliminate the oxidases and in particular polyphenoloxidase. It is, for example, possible to filter the extract of the invention through a dialysis membrane in order to remove molecules with a molecular weight of greater than 100,000 daltons therefrom. It is also possible to subject the extract to fractionation by selective precipitations.

Other techniques make it possible to avoid the aforesaid oxidation phenomena. In particular, the extract can also be stabilized. Any known stabilization method can be used according to the invention. It is also possible, for example, to stabilize the extract of the invention by bubbling nitrogen therethrough in order to eliminate the dissolved oxygen, or by adding cysteine and/or sulfur-containing derivatives thereto, at a final concentration of from 0.5 g/l to 10 g/l, and preferably from 1 g/l to 2.5 g/l.

Of course, the extract according to the invention can be fractionated and stabilized.

The extract can, by itself, constitute the active principle of the compositions of the invention.

In a particular embodiment of the invention, the extract does not contain any rosmarinic acid.

One type of preparation of an extract which can be used according to the invention is given, moreover, in the examples to follow.

The amount of extract administered according to the invention depends, of course, on the desired effect, and may therefore vary over wide limits.

To provide an order of magnitude, an extract as described above can be administered in an amount representing from 0.001% to 20% of the total weight of the composition (formulated into a vehicle, diluent or carrier therefor), and preferably in an amount representing from 0.01% to 10% of the total weight of the composition.

The present invention also features a regime/regimen for the cosmetic treatment of the skin, to stimulate collagen synthesis and/or treat cutaneous disorders related to age and/or to menopause and/or to combat thinning of the dermis and/or to combat the appearance of flabby and/or wrinkled skin, comprising topically applying at least one extract of at least one plant of the Rosmarinus genus, or composition comprised thereof, onto the skin, onto the hair and/or onto the mucous membranes.

The cosmetic regime/regimen according to the present invention is carried out for such period of time as required to elicit the desired therapeutic effect, by topically applying the subject compositions whether formulated, for example, as creams, gels, serums, lotions, milks, shampoos or sunscreen compositions, onto the skin or on the hair, or application of a dentrifrice onto the gums.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

Preparation of an Extract of Rosmarinus officinalis

636 grams of plant material from Rosmarinus officinalis, the part exposed to the air corresponding to stems and leaves, were immersed in approximately 1 kg of liquid nitrogen.

After removal of the stems, the leaves were reduced to powder by grinding in a Turrax at 24,000 rpm for 1 minute at 4° C. (ice bath).

The powder obtained was mixed with 5 liters of 0.05 M phosphate buffer at pH 8.5.

The mixture was stirred for 30 minutes at 4° C., and then centrifuged at 10,000 G at 4° C.

The supernatant was filtered through 0.22 (m (sterilizing filtration).

The extract was then fractionated by ultrafiltration through a membrane of the Sartorius type in order to eliminate oxidation phenomena therefrom.

The extract was then lyophilized. 29.5 grams of active extract termed “lyophilized extract” were thus obtained.

### EXAMPLE 2

Effect of the Extract of Example 1 on Collagen Synthesis

The study was conducted by measuring the incorporation of radioactive proline into cultures of normal human dermal fibroblasts.

The fibroblast cultures were prepared according to conventional cell culture methods, i.e., in MEM/M199 medium marketed by Gibco in the presence of sodium bicarbonate (1.87 mg/ml), of L-glutamine (2 mM), of penicillin (50 IU/ml) and of 10% of fetal calf serum (Gibco).

The assay was carried out on cell cultures at 80% confluency, in a 24-well plate. The extract of Example 1 (lyophilized extract titrated at 25 mg/ml of water to obtain a stock solution at 100%), at the final concentrations of 0.2%, 1% and 2%, was contacted with the cells for 48 hours. The labeling with tritiated proline (L-[2,3-³H]-proline sold by Amersham, 33 (μCi/ml) was carried out for 24 hours.

The level of tritiated proline incorporated was measured at the end of the assay by acid precipitation of the proteins on filters and liquid scintillation counting.

The results were evaluated with respect to a control consisting of cells which had not been treated with the extract of Example 1.

A positive control (vitamin C at 20 (g/ml) known to stimulate collagen synthesis was introduced into the assay as a reference.

The results of this assay, expressed as percentages of activation, are reported in the following Table I:

TABLE I

| Treatment | cpm | % | p |
|---|---|---|---|
| Untreated cells | 7498 | — | — |
| Vitamin C | 13091 | 75 | <0.01 |
| Extract of Example 1 | | | |
| 0.2% | 9537 | 8 | >0.05 |
| 1% | 9181 | 22 | >0.05 |
| 2% | 8098 | 27 | >0.05 |

cpm: counts per minute
p: confidence interval calculated according to Dunett's method.

These results evidence that the extract of Example 1 significantly stimulated the incorporation of proline into the collagen and, therefore, that it exhibited a positive effect on collagen neosynthesis.

### EXAMPLE 3

Effect of the Extract of Example 1 on the Expression of Collagenases

The study permitted evaluation of the effectiveness of a product in inhibiting the induction of transcription of the collagenase gene by ultraviolet radiation.

The assay was carried out on HeLa cells (ATCC CCL2) into which the promoter of the collagenase 1 gene (3.8 kB) cloned upstream of the chloramphenicol acetyltransferase (CAT) gene had been introduced.

The cells were exposed to ultraviolet B (UVB) radiation emitted by a Philips TL 12W/20 tube, with Kadacel filters to remove the UVCs, at the doses of 5 and 10 mJ/cm². The cells were then contacted, for 2 hours, with the extract of Example 1 at doses ranging from 0.05% to 0.5%.

The CAT was assayed using a “CAT-Elisa” kit marketed by Boehringer, according to the supplier's indications.

A “base level” of expression of the CAT was measured in cells which had undergone neither ultraviolet radiation nor treatment with the test product.

The results of the assay, reported in the following Table II, are expressed relative to the base level (100%), as percentage of activation or inhibition of CAT gene expression.

These results therefore reflect the activity of the collagenase 1 promoter after induction by ultraviolet rays and in the presence or absence of the test product.

TABLE II

| UV B* | No Extract | Extract at 0.05% | Extract at 0.1% |
|---|---|---|---|
| 5 | 926 | 416 | 289 |
| 10 | 3930 | 1104 | 568 |

*Dose in mJ/cm²

These results evidenced that:

(a) the activity of the collagenase promoter was stimulated by UVB rays (“no extract” column);

(b) this stimulation was decreased in the presence of the extract of Example 1 (“Extract at . . . ” columns).

These results clearly indicate the inhibitory effect on the synthesis of collagenase 1, by inhibition of the activity of its promoter, of the extract from Rosmarinus officinalis, of Example 1.

### EXAMPLES 4-12

The following Examples 4-12 are of specific compositions formulated as indicated, and comprising at least one extract from Rosmarinus officinalis. These compositions were formulated simply by intimately admixing the various components thereof.

### EXAMPLE 4

Composition 1—Makeup Removing Lotion for the Face

| | | |
|---|---|---|
| Extract from Example 1 | | 10.00% |
| Antioxident | | 0.05% |
| Isopropanol | | 40.00% |
| Preservative | | 0.30% |
| Water | q.s. for | 100.00% |

### EXAMPLE 5

Composition 2—Shampoo

| | | |
|---|---|---|
| Extract of Example 1 | | 0.50% |
| Hydroxypropylcellulose | | 1.00% |
| (Klucel H ® marketed by Hercules) | | |
| Fragrance | | 0.50% |
| Preservative | | 0.30% |
| Water | q.s. for | 100.00% |

### EXAMPLE 6

Composition 3—Facial Care Cream (Oil-in-water Emulsion)

| | | |
|---|---|---|
| Extract of Example 1 | | 2.00% |
| Glyceryl stearate | | 2.00% |
| Polysorbate 60 (Tween 60 ® marketed | | 1.00% |
| by ICI) | | |
| Stearic acid | | 1.40% |
| Triethanolamine | | 0.70% |
| Carbomer | | 0.40% |
| Liquid fraction of karite butter | | 12.00% |
| Perhydrosqualene | | 12.00% |
| Antioxidant | | 0.05% |
| Fragrance | | 0.50% |
| Preservative | | 0.30% |
| Water | q.s. for | 100.00% |

### EXAMPLE 7

Composition 4—Gel for the Skin

| | | |
|---|---|---|
| Extract of Example 1 | | 0.50% |
| All-trans-retinoic acid | | 0.05% |
| Hydroxypropylcellulose | | 1.00% |
| (Klucel H ® marketed by Hercules) | | |
| Antioxidant | | 0.05% |
| Isopropanol | | 40.00% |
| Preservative | | 0.30% |
| Water | q.s. for | 100.00% |

### EXAMPLE 8

Composition 5—Face Care Gel

| | | |
|---|---|---|
| Extract of Example 1 | | 5.00% |
| Hydroxypropylcellulose | | 1.00% |
| (Klucel H ® marketed by Hercules) | | |
| Antioxidant | | 0.05% |
| Isopropanol | | 40.00% |
| Preservative | | 0.30% |
| Water | q.s. for | 100.00% |

### EXAMPLE 9

Composition 6—Gel

| | | |
|---|---|---|
| Extract of Example 1 | | 1.00% |
| Hydroxypropylcellulose | | 1.00% |
| (Klucel H ® marketed by Hercules) | | |
| Antioxidant | | 0.05% |
| Lidocaine hydrochloride | | 2.00% |
| Isopropanol | | 40.00% |
| Preservative | | 0.30% |
| Water | q.s. for | 100.00% |

### EXAMPLE 10

Composition 7—Sunburn Care Cream (Oil-in-water Emulsion)

| | | |
|---|---|---|
| Extract of Example 1 | | 2.50% |
| Glyceryl stearate | | 2.00% |
| Polysorbate 60 (Tween 60 ® marketed | | 1.00% |
| by ICI) | | |
| Stearic acid | | 1.40% |
| Glycyrrhetinic acid | | 2.00% |
| Triethanolamine | | 0.70% |
| Carbomer | | 0.40% |
| Liquid fraction of karite butter | | 12.00% |
| Sunflower oil | | 10.00% |
| Antioxidant | | 0.05% |
| Fragrance | | 0.50% |
| Preservative | | 0.30% |
| Water | q.s. for | 100.00% |

### EXAMPLE 11

Composition 8—Antiwrinkle Care Cream for the Face (Oil/water Emulsion)

| | | |
|---|---|---|
| Extract of Example 1 | | 5.00% |
| Glyceryl stearate | | 2.00% |
| Polysorbate 60 (Tween 60 ® marketed | | 1.00% |
| by ICI) | | |
| Stearic acid | | 1.40% |
| 5-(n-octanoyl)salicylic acid | | 0.50% |
| Triethanolamine | | 0.70% |
| Carbomer | | 0.40% |
| Liquid fraction of karite butter | | 12.00% |
| Perhydrosqualene | | 12.00% |
| Antioxidant | | 0.05% |
| Fragrance | | 0.50% |
| Preservative | | 0.30% |
| Water | q.s. for | 100.00% |

### EXAMPLE 12

Composition 9—Lotion

| | | |
|---|---|---|
| Extract of Example 1 | | 0.50% |
| Glycolic acid | | 50.00% |
| Hydroxypropylcellulose (Klucel H ® | | 0.05% |
| marketed by Hercules) | | |
| Preservative | | 0.30% |
| NaOH | q.s. for | pH = 2.8 |
| Ethanol | q.s. for | 100.00% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime/regimen for inhibiting/treating the adverse signs or effects of cutaneous aging, comprising topically applying onto the skin, hair and/or mucous membranes of an individual subject in need of such treatment, for such period of time as required to elicit the desired response, a thus effective amount of at least one extract of at least one plant of the Rosmarinus genus, or cosmetic/dermatological composition comprised thereof.

2. A regime/regimen for stimulating collagen synthesis in the skin, hair and/or mucous membranes of an individual subject in need of such treatment, comprising topically applying onto such skin, hair and/or mucous membranes, for such period of time as required to elicit the desired response, a thus effective amount of at least one extract of at least one plant of the Rosmarinus genus, or cosmetic/dermatological composition comprised thereof.

3. A regime/regimen for inhibiting the expression of extracellular matrix proteases in the skin, hair and/or mucous membranes of an individual subject in need of such treatment, comprising topically applying onto such skin, hair and/or mucous membranes, for such period of time as required to elicit the desired response, a thus effective amount of at least one extract of at least one plant of the Rosmarinus genus, or cosmetic/dermatological composition comprised thereof.

4. The regime/regimen as defined by claim 3, for inhibiting the expression of metalloproteinases.

5. The regime/regimen as defined by claim 4, for inhibiting the expression of type 1 metalloproteinases.

6. The regime/regimen as defined by claim 1, comprising inhibiting/treating the adverse signs or effects of cutaneous aging related to menopause.

7. The regime/regimen as defined by claim 1, for combating skin wrinkles and fine lines.

8. The regime/regimen as defined by claim 1, for combating withered skin.

9. The regime/regimen as defined by claim 1, for combating flabby skin.

10. The regime/regimen as defined by claim 1, for combating thinned skin.

11. The regime/regimen as defined by claim 1, for combating lack of elasticity and/or loss of tone of the skin.

12. The regime/regimen as defined by claim 1, for combating skin internally damaged from exposure to ultraviolet radiation.

13. The regime/regimen as defined by claim 1, for combating damaged skin collagen.

14. The regime/regimen as defined by claim 1, said at least one extract of at least one plant of the Rosmarinus genus being obtained from a whole plant, or from a part thereof.

15. The regime/regimen as defined by claim 14, said at least one extract of at least one plant of the Rosmarinus genus being obtained from the leaves, stems, flowers, petals, roots, and/or dedifferentiated cells thereof.

16. The regime/regimen as defined by claim 15, said at least one extract of at least one plant of the Rosmarinus genus being obtained from the leaves thereof.

17. The regime/regimen as defined by claim 1, said at least one extract of at least one plant of the Rosmarinus genus being obtained by culturing in vitro.

18. The regime/regimen as defined by claim 1, said at least one extract of at least one plant of the Rosmarinus genus being obtained by culturing in vivo.

19. The regime/regimen as defined by claim 1, said at least one extract of at least one plant of the Rosmarinus genus comprising an aqueous extract.

20. The regime/regimen as defined by claim 1, said at least one extract of at least one plant of the Rosmarinus genus being devoid of rosmarinic acid.

21. A topically applicable cosmetic/dermatological composition for inhibiting/treating the adverse signs or effects of cutaneous aging, comprising a thus effective amount of at least one extract of at least one plant of the Rosmarinus genus, formulated into a topically applicable, cosmetically/dermatologically acceptable vehicle, diluent or carrier therefor.

22. The topically applicable cosmetic/dermatological composition as defined by claim 21, formulated as an emulsion, cream, gel, lotion, milk, shampoo, serum, sunscreen, or dentifrice.

23. The topically applicable cosmetic/dermatological composition as defined by claim 22, devoid of rosmarinic acid.

24. The topically applicable cosmetic/dermatological composition as defined by claim 22, comprising from about 0.001% to about 20% by weight of said at least one extract of at least one plant of the Rosmarinus genus.

25. The topically applicable cosmetic/dermatological composition as defined by claim 22, comprising from about 0.01% to about 10% by weight of said at least one extract of at least one plant of the Rosmarinus genus.

26. The topically applicable cosmetic/dermatological composition as defined by claim 21, said at least one extract of at least one plant of the Rosmarinus genus having been lyophilized, fractionated and/or stabilized.

27. The topically applicable cosmetic/dermatological composition as defined by claim 21, said at least one extract of at least one plant of the Rosmarinus genus having been stripped of any species having a molecular weight greater than 100,000 daltons.

28. The topically applicable cosmetic/dermatological composition as defined by claim 21, said at least one extract of at least one plant of the Rosmarinus genus comprising a species of Rosmarinus tenuifolius or Rosmarinus latifolius.
